(19) United States
(12) Patent Application Publication
(10) Pub. No.: US 2014/0283153 A1
(43) Pub. Date: Sep. 18, 2014

(54) TRANSGENIC ANIMALS AND METHODS OF USE

(71) Applicant: Trianni, Inc., Emeryville, CA (US)

(72) Inventor: Nigel Killeen, Emeryville, CA (US)

(21) Appl. No.: 14/353,224

(22) PCT Filed: Oct. 26, 2012
(86) PCT No.: PCT/US12/62118

§ 371 Date: Apr. 21, 2014

Related U.S. Application Data
(60) Provisional application No. 61/553,147, filed on Oct. 28, 2011.

(51) Int. Cl. A01K 67/027

(52) U.S. Cl. CPC A01K 67/0275
USPC 800/9

(57) ABSTRACT

This invention relates to transgenic vertebrates, and more specifically to transgenic vertebrates for the development of human therapeutics.

### CROSS-REFERENCE TO RELATED APPLICATIONS

This application claims the benefit of provisional U.S. Patent Application No. 61/553,147 filed on Oct. 28, 2012.

### FIELD OF THE INVENTION

This invention relates to transgenic vertebrates, and more specifically to transgenic vertebrates for the development of human therapeutics.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Monoclonal antibodies are a class of biologic therapeutic that has enjoyed remarkable success in the clinic during the past decade. As a consequence of this, by 2007 monoclonal antibodies had assumed the top revenue-generating position among biologic therapeutics, and by 2010 worldwide sales exceeded 45 billion US dollars. There are currently over 200 monoclonal antibodies being tested in clinical trials making clear that revenues are likely to continue to increase for many years.

Despite their successes, monoclonal antibodies have not been able to address certain areas of unmet clinical need. Therapeutic development failure of monoclonal antibodies can be due to a lack of desired targeting specificity or less than optimal therapeutic properties, such as poor tissue penetration or too short of a half-life in the system. Because of this, there has been considerable interest in the biotechnology industry in developing alternative biologic therapeutics with improved properties for targets that are intractable to monoclonal antibodies.

During the last decade in particular, there have been impressive successes in isolating alternative biologics with potentially beneficial therapeutic properties. Nonetheless, the in vitro procedures used to identify and develop these molecules typically fall short of replicating the power of an in vivo immune system in terms of coupling a highly effective capability for diversification with rapid and similarly effective selection for optimal target-binding properties.

Based on the foregoing, it is clear that there is a need in the art for nonhuman host animals having the ability to produce and extensively diversify antigen-binding proteins that are alternatives to antibodies, and transgenic non-human animals having the ability to produce such antigen-binding proteins.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides non-human host cells and animals for the identification and production of biologic therapeutics that selectively bind to one or more antigens of interest. These biologics resemble antibodies functionally, in that they have antigen-binding capacity and can be diversified allowing for recognition of many different antigens; they differ from antibodies, however, in the primary amino acid sequence of their antigen-binding domains, and consequently in their antigen-binding properties. They are of interest because they represent alternatives to antibodies and may be better suited than antibodies for particular applications in diagnostic, therapeutic, or research contexts. They are referred to herein as “alternative biologics” in consideration of the foregoing. The cells and animals of the present invention provide an enhanced tool for identifying and optimizing such alternative biologics.

The tools of the invention are created by engineering the immune systems of vertebrate animals such that they express the biologic therapeutics in place of, or in addition to antibodies. By harnessing the immune system, the animals and methods of the invention provide an in vivo mechanism for coupling a highly effective and focused genome diversification capability with rapid and similarly effective cell-based selection for optimal target-binding properties. The modified genomes of the animals of the invention allow large-scale diversification of the biologic therapeutics by a genomic rearrangement process similar to the process used to diversify antibodies in vertebrates such as rodents, or a gene conversion process similar to that used to diversify antibodies in chickens.

Specifically, the present invention comprises non-human vertebrate cells and non-human vertebrates having modified genomes comprising nucleic acids encoding protein “scaffolds”, where such scaffolds comprise an epitope-binding site, or sites, and consequently have the capacity to bind to epitopes on antigens. The nucleic acid encoding the scaffold with its associated epitope-binding site(s) is embedded within a gene encoding an immunoglobulin chain such that it replaces the part of the gene that encodes the antigen-binding domain of the immunoglobulin. In one preferred form of the invention, the immunoglobulin chain is a heavy chain and the antigen-binding domain that is replaced is the amino-terminal variable domain. This replacement results in a chimeric antibody-like molecule with a scaffold domain comprising the antigen-binding domain but with the rest of the molecule being comprised of the heavy chain constant domain.

In a preferred version of the invention, the nucleic acid encoding the scaffold and its associated epitope-binding site(s) is introduced into the genome within an endogenous immunoglobulin gene such as the gene encoding immunoglobulin heavy chains. The insertion is accompanied or preceded by a deletion/inactivation event in which the endogenous variable (V) region gene segments and/or diversity (D) and joining (J) gene segments are eliminated from the gene or otherwise rendered inactive. The inserted exogenous DNA is engineered such that it can participate in RAG-1/RAG-2-mediated recombination, i.e., in a version of V(D)J recombination in which the immunoglobulin V, D, and J components are replaced with those that encode the scaffold and its epitope-binding site(s) after recombination. RAG-1/RAG-2-mediated V(D)J recombination is a primary means for diversifying antibody genes during B cell development, and such recombination is harnessed here as a means for similarly diversifying the scaffold-encoding genes. Alternatively, the inserted DNA is engineered such that it participates efficiently in gene conversion as a means for accomplishing diversity. Gene conversion is used as the primary means for diversifying antibody genes in avian species, and gene conversion thus is the preferred mechanism for diversifying genes encoding scaffold domains in such species.

In preferred aspects of the invention, the nucleic acid encoding the scaffold is generated by genomic recombination between two or more distinct arrays of scaffold gene segments. In the case where three arrays are employed, the 5′ array comprises gene segments that resemble V gene segments in that they are flanked by a recombination signal sequence (RSS) on their 3′ ends (this type of array is hereafter referred to as “V-like”, reflecting principally where the RSSs are placed in the array). The middle array comprises gene segments that resemble D gene segments in being flanked on both ends by RSSs (this type of array is hereafter referred to as “D-like”). The 3′ array comprises gene segments that resemble J gene segments in being flanked on their 5′ ends by RSSs (this type of array is hereafter referred to as “J-like”). Each array would comprise a minimum of one gene segment.

In an embodiment where two arrays of gene segments are used to generate the open reading frame encoding the scaffold, a V-like and J-like array preferably may be employed without an intervening D-like array.

In an embodiment where more than three arrays of gene segments are used to generate the open reading frame encoding the scaffold, more than one intervening D-like array preferably may be employed.

In the case where diversification is accomplished primarily, if not exclusively, by gene conversion, such as in avian species, a scaffold-encoding gene segment is placed downstream of an array of pseudo-gene segments. Here, the array serves as a reservoir of donor DNA sequences that are drawn upon during gene conversion to diversify the sequence of the downstream scaffold-encoding gene segment.

In a preferred embodiment of the invention, the protein sequence of the scaffold domain is based on a natural sequence, but contains modifications designed to enhance its capacity to bind to antigens and/or to enhance the scaffold in other ways, such as, but not limited to, improvements in stability, improvements in amenability to bioprocessing and manufacturing, improvements in pharmacodynamics and other aspects of in vivo function, and improvements in antigenicity that render it less likely to be recognized as foreign by the immune systems of humans or other species.

In one embodiment, the invention comprises gene segments that although based on a natural sequence, are otherwise designed and do not occur in nature. In other embodiments some of the gene segments, or indeed arrays of gene segments, are natural in origin. For example, arrays of antibody D and/or J gene segments are employed for the purpose of diversifying a specific region of a scaffold domain.

A feature of the invention is the diversification of the protein sequence of the scaffold domain by the natural process that normally diversifies the genes encoding antibodies, i.e., V(D)J recombination and/or gene conversion. The diversification process results in individual B lymphocytes containing scaffold-encoding genes that have been independently diversified during the development of the cells from hematopoietic progenitor cells. Furthermore, the introduced scaffold-encoding gene segments are arranged in such a manner that V(D)J recombination and/or gene conversion results in diversification of the antigen-binding sites of the encoded scaffold domains. In the case of diversification by V(D)J recombination, the antigen-binding sites are encoded by pieces of DNA that include the junctions generated by joining V-like gene segments with D-like or J-like gene segments, or by joining D-like gene segments with J-like gene segments and/or with other intervening D-like gene segments. Diversity is accomplished in this context either through usage of different members of the V-like, D-like or J-like arrays, or by imprecision/variation in the recombination process, such as is typical during V(D)J recombination.

The scaffold domains that are expressed from the introduced, exogenous DNA may comprise one or more discrete antigen-binding regions, each of which may be independently diversified during V(D)J recombination. That is, each of the regions may be encoded by pieces of DNA that are independently diversified by usage of different members of a gene segment array and/or by imprecision/variation in the junctions between recombined gene segments. The different antigen-binding regions may engage the same antigen, or they may have specificity for distinct antigens.

Alternatively, the antigen-binding region of a scaffold domain may be comprised of protein features that are separated from one another in the primary protein sequence yet are brought close to one another in the tertiary protein structure. Each of these features may again derive from pieces of DNA that are independently diversified by usage of different members of a gene segment array and/or by imprecision/variation in the junctions between recombined gene segments.

In one alternative embodiment of the invention, instead of inserting the scaffold-encoding gene segments into the endogenous locus encoding the immunoglobulin heavy chain, the donor DNA is placed in a locus encoding an immunoglobulin light chain. In this case, the modified light chain locus would express the scaffold domain as part of a chimeric molecule comprising a carboxy-terminal light chain constant domain. Alternatively, the inserted DNA may also include genomic DNA, or a semi- or fully-recombinant or synthetic version of genomic DNA, encoding immunoglobulin heavy chain constant domains. As is true of the endogenous heavy chain locus, the inserted constant domain DNA may retain the capacity to undergo class-switching allowing for expression of scaffold domains as part of chimeric heavy chains of different isotypes. Alternatively, a smaller piece of DNA may be employed such that the class-switching capacity is not included resulting in expression of only one isotype of constant domain from the scaffold-encoding locus.

In a further embodiment of the invention, the scaffold-encoding gene segments may be expressed from a locus that comprises mutated heavy chain constant domains. The mutations may influence the expression of the constant domains or influence their function. One specific class of mutations influences association between the encoded heavy chain constant domains and chaperone proteins and/or light chain proteins. The effect of the mutations is to interfere with such associations or abrogate them and thus allows for cell surface expression or secretion of the scaffold-domain-containing chimeric immunoglobulin molecules without associated light chains.

In a further embodiment, the scaffold-encoding gene segments are inserted into a locus in the genome that does not encode immunoglobulin chains. In this case, expression of the scaffold-encoding locus in B lymphocytes would be accomplished through inclusion of necessary cis-acting sequences, such as enhancers and promoters derived from an immunoglobulin gene, and/or from other genes that are normally expressed during B lymphocyte development.

In a preferred embodiment of the invention, the scaffold-encoding DNA introduced into the genome is comprised of noncoding sequences derived themselves from the recipient genome. These noncoding sequences include introns, intergenic sequences (and/or sequences from between gene segments), untranslated regions, promoters, other transcriptional regulatory sequences, recombination signal sequences or other sequences that influence V(D)J recombination or gene conversion, and sequences involved in regulating chromatin structure and accessibility. Alternatively, the noncoding sequences are derived from other species, such as vertebrate species related to the recipient animal, or they may be partially or entirely designed/artificial.

In a preferred embodiment of the invention where there is expression of the scaffold-encoding gene from a locus other than the endogenous gene that encodes the immunoglobulin heavy chain, the invention may also feature mutation or inactivation of the endogenous heavy chain locus. B lymphocyte development is prevented by inactivation of the heavy chain locus. Inactivation is advantageous because it allows for B cell development to be rendered dependent on expression of a surrogate for the endogenous heavy chain, specifically, a chimeric heavy chain comprised of an antigen-binding scaffold domain fused to heavy chain constant domains. Thus, inactivation of the endogenous heavy chain locus establishes developmental selection for lymphocytes that express the exogenous scaffold-containing chimeric molecules. Alternatively, B cells are made to express a form of the heavy chain molecule that is largely, if not entirely, deprived of antigen-binding variability in its amino-terminal domain. In this latter case, the heavy chain molecule is engineered to retain dependence on light chain association for surface expression or secretion, and thus the molecule is available for dimerization with a scaffold domain-containing chimeric molecule comprised of a light chain constant domain.

In yet another preferred embodiment of the invention, the scaffold-encoding gene segments are inserted into the genome in an arrangement that does not immediately allow for expression of a scaffold domain as part of a chimeric immunoglobulin molecule. Such expression only occurs after diversification of the gene by V(D)J rearrangement. In combination with inactivation of the endogenous heavy chain gene, expression only after diversification creates a situation in which there is developmental selection for B lymphocytes that have undergone productive V(D)J rearrangement and thus diversification of the scaffold-encoding locus. In a similar fashion, cellular selection could be influenced by, or rendered entirely dependent on, diversification of the scaffold-encoding locus by gene conversion.

In some aspects, the protein scaffold is derived from CTLA-4, in other aspects, the protein scaffold is derived from lipocalin, anticalin, Protein A, Protein G, fibronectin, an A domain, a Heat shock protein, GroEI, GroES, transferrin, an ankyrin repeat protein, a peptide aptamer, a C-type lectin domain, a human γ-crystallin, ubiquitin, a PDZ domain, a scorpion toxin, a kunitz-type domain of human protease inhibitors, fibronectin, an affibody, an ankyrin repeat protein or an adnectin.

In some aspects, the genome that is modified by insertion of the scaffold-encoding gene segments is a vertebrate nonhuman mammal, and preferably the mammal is a rodent, e.g., a mouse or rat. In other aspects, the vertebrate is avian, e.g., a chicken.

In one specific aspect, the invention provides a method for generating a non-human transgenic vertebrate cell comprising a chimeric immunoglobulin nucleic acid sequence, the method comprising: a) introducing two or more recombinase targeting sites into a non-human vertebrate cell and integrating at least one site in the cell's genome upstream and at least one site downstream of a genomic region comprising that part of an endogenous immunoglobulin gene that comprises the V and J gene segments, and the D gene segments if they are also present in the locus (this part of the locus is hereafter referred to collectively as the “variable region”); and b) introducing a nucleic acid sequence comprising scaffold-encoding gene segments with associated noncoding DNA into the non-human vertebrate host cell via recombinase-mediated cassette exchange (RMCE). In a preferred aspect, the method further provides deleting the endogenous immunoglobulin variable region flanked by the two introduced recombinase sites prior to step b) or simultaneously with it.

Preferably, the scaffold-encoding DNA is introduced into the non-human vertebrate host cell as a single nucleic acid region. It may also be introduced to the vertebrate host cell in discrete segments.

In a preferred version of the invention, the insertion of the DNA comprising scaffold-encoding gene segments is accomplished by RMCE. The DNA to be inserted is flanked on both ends by binding sites for a single site-specific recombinase, such as the Cre, Flp or Dre recombinase. Optimally, the two sites differ from one another such that they remain substrates for the recombinase and will undergo efficient homotypic recombination, but will not participate efficiently in heterotypic recombination (i.e., they will not efficiently recombine with one another). RMCE (i.e., the recombinase-catalyzed exchange of pieces of DNA between a donor and a recipient DNA molecule) can occur if a donor piece of DNA (in this invention, this is the DNA that contains the scaffold-encoding gene segments) is flanked by the same recombinase recognition sites that are present in the recipient location (in a preferred version of this invention, this is the variable region of an endogenous immunoglobulin locus). Deletion of the variable region of an immunoglobulin locus can be a direct byproduct of RMCE, or deletion can be accomplished in advance (or even during the same genomic modification step) if the variable region is flanked by recombinase recognition sites that permit homotypic recombination. RMCE requires the presence of the recombinase in the cell at the same time as the DNA to be inserted into the genome is also present. This can be accomplished by delivering a recombinase expression vector into the cells with the donor DNA, or by delivering the recombinase protein to the cells. Similarly, the recombinase-mediated deletion of a genomic interval (such as the variable region of an immunoglobulin locus) also requires delivering the recombinase into the cell via the same types of procedures.

Thus, in another aspect, the invention provides methods for generating a non-human transgenic vertebrate cell comprising a chimeric immunoglobulin nucleic acid sequence, the method comprising: a) introducing two or more site-specific recombination sites that are not capable of recombining with one another into the genome of a cell of a non-human vertebrate host, where at least one recombination site is introduced upstream of an endogenous immunoglobulin variable region locus and at least one recombination site is introduced downstream of the endogenous immunoglobulin variable region locus; b) providing to the host cell a vector comprising scaffold-encoding gene segments with associated noncoding DNA, said nucleic acid sequence comprising: i) gene segments containing coding sequences for scaffold domains arranged in such a fashion that they permit diversification by V(D)J recombination and/or gene conversion and ii) non-coding sequences that may be based on an endogenous immunoglobulin variable region locus of the non-human vertebrate host or that of an alternative locus or host, and (iii) two site-specific recombination sites flanking the coding sequences and non-coding sequences, where the two site-specific recombination sites are the same as those that flank the endogenous variable immunoglobulin region of the host cell of a); c) introducing the vector of step b) and a site-specific recombinase capable of recognizing the two recombinase sites to the cell; d) allowing a recombination event to occur between the genome of the cell of a) and the introduced nucleic acid, resulting in a replacement of the endogenous immunoglobulin variable region locus with the DNA comprising scaffold-encoding gene segments.

In another specific aspect of this method, the method further provides deleting the endogenous immunoglobulin variable region locus flanked by the two introduced recombinase sites prior to step c).

The invention provides yet another method for generating a transgenic non-human vertebrate cell, said method comprising: a) providing a non-human vertebrate cell having a genome that comprises two sets of site-specific recombination sites that are not capable of recombining with one another, and which flank a portion of an endogenous immunoglobulin region of the host genome; b) deleting the portion of the endogenous immunoglobulin variable region locus of the genome by introduction of a recombinase that recognizes a first set of site-specific recombination sites, wherein such deletion in the genome retains the second set of site-specific recombination sites; c) providing a vector comprising scaffold-encoding gene segments with associated noncoding DNA, and a gene, or part of a gene, encoding a drug resistance protein; d) introducing the vector of step c) and a site-specific recombinase capable of recognizing the second set of recombinase sites to the cell; e) allowing a recombination event to occur between the genome of the cell and the introduced nucleic acid sequence, resulting in a replacement of the endogenous immunoglobulin variable region locus with the DNA comprised of scaffold-encoding gene segments; and f) selecting for cells that have undergone the recombination event using a drug that is appropriate for use as a selection agent with the introduced drug resistance gene.

Preferably, the non-human mammalian cell for use in each of the above methods is a mammalian cell, and more preferably a mammalian embryonic stem (ES) cell. In other aspects, the cell may be an avian cell, and preferably an avian primordial germ cell.

Once the cells have been subjected to the replacement of the endogenous immunoglobulin variable region nucleic acid sequence, cells comprising the introduced scaffold-encoding nucleic acid sequence are preferably isolated. In a preferred aspect of the invention, the cells are non-human mammalian embryonic stem (ES) cells, and the isolated ES cell is then utilized to create a transgenic non-human mammal expressing the chimeric immunoglobulin variable nucleic acid sequence. In other aspects, the cells are primordial germ cells, and the isolated germ cell is then utilized to create a transgenic bird expressing the chimeric immunoglobulin variable region.

In some aspects, the cells and transgenic animals of the present invention are used as research tools.

In a specific aspect, the invention provides a method for generating a nonhuman transgenic mammalian cell comprising a chimeric immunoglobulin nucleic acid sequence, said method comprising: a) providing a non-human mammalian embryonic stem (ES) cell having a genome that contains two site-specific recombination sites that are not capable of recombining with each other, and which flank a portion of an endogenous immunoglobulin region; b) providing a vector comprising gene segments encoding scaffold domains flanked by the same two site-specific recombination sites that flank the portion of the endogenous immunoglobulin region in the ES cell; c) bringing the ES cell and the vector into contact with a site-specific recombinase capable of recognizing the two recombinase sites under appropriate conditions to promote a recombination event resulting in the replacement of a portion of the endogenous immunoglobulin region with the chimeric immunoglobulin nucleic acid sequence in the ES cell.

In another aspect, the invention provides a method for generating a transgenic non-human mammal comprising a chimeric immunoglobulin region, the method comprising: a) introducing one or more site-specific recombination sites that are not capable of recombining with one another into the genome of a cell of a non-human vertebrate host; b) providing a vector comprising scaffold-encoding gene segments with associated noncoding DNA; c) introducing the vector of step b) and a site-specific recombinase capable of recognizing one set of the one or more site-specific recombinase sites to the cell; d) allowing a recombination event to occur between the genome of the cell of a) and the introduced DNA, resulting in a replacement of the endogenous immunoglobulin variable region with the scaffold-encoding DNA; e) selecting a cell which comprises the chimeric immunoglobulin nucleic acid sequence; and f) utilizing the cell to create a transgenic animal comprising the chimeric immunoglobulin nucleic acid sequence.

The invention provides yet another method for generating a transgenic nonhuman animal comprising a chimeric immunoglobulin nucleic acid sequence, said method comprising: a) providing a non-human vertebrate cell having a genome that comprises two sets of site-specific recombination sites that are not capable of recombining with one another and that flank a portion of an endogenous immunoglobulin variable region locus of the host genome; b) deleting the portion of the endogenous immunoglobulin region locus of the host genome by introduction of a recombinase that recognizes a first set of site-specific recombination sites, wherein such deletion in the genome retains the second set of site-specific recombination sites; c) providing a vector comprising scaffold-encoding gene segments with associated noncoding DNA; d) introducing the vector of step c) and a site-specific recombinase capable of recognizing a second set of site-specific recombination sites to the cell; e) allowing a recombination event to occur between the genome of the cell and the introduced DNA resulting in a replacement of the endogenous immunoglobulin region with the scaffold-encoding DNA; f) selecting a cell that comprises the chimeric immunoglobulin sequence; and g) utilizing the cell to create a transgenic animal comprising the chimeric immunoglobulin variable nucleic acid sequence.

The invention provides yet another method for generating a transgenic non-human mammal comprising a chimeric immunoglobulin region, said method comprising: a) providing a non-human mammalian embryonic stem (ES) cell having a genome that contains two site-specific recombination sites that are not capable of recombining with each other, and that flank a portion of the endogenous immunoglobulin variable region locus; b) providing a vector comprising scaffold-encoding gene segments with associated noncoding DNA; c) bringing said ES cell and said vector into contact with a site-specific recombinase capable of recognizing the two recombinase sites under appropriate conditions to promote a recombination event resulting in the replacement of a portion of the endogenous immunoglobulin region locus with the introduced DNA in the ES cell; d) selecting an ES cell that comprises the chimeric immunoglobulin nucleic acid sequence; and e) utilizing the cell to create a transgenic animal comprising the chimeric immunoglobulin gene locus.

In a specific aspect of the invention, the transgenic non-human vertebrates are mammals, and preferably the mammals are rodents, e.g., a mouse or a rat. In other aspects, the transgenic non-human vertebrates are avian, e.g., a chicken.

In yet another embodiment, the present invention provides a transgenic non-human vertebrate with a genome comprising an introduced region, said introduced region comprising a nucleic acid encoding one or more antigen-binding sites associated with a protein scaffold; and in yet another embodiment, the present invention provides a transgenic non-human vertebrate with a genome comprising an introduced region, said introduced region comprising a nucleic acid encoding one or more protein scaffolds each associated with one or more antigen-binding sites.

In some aspects of these embodiments of the invention, the protein scaffold is derived from CTLA-4, lipocalin, anticalin, Protein A, an affibody, Protein G, fibronectin, adnectin an A-domain, a Heat shock protein, GroEI, GroES, transferring, ankyrin, a C-type lectin domain, human γ-crystallin, human ubiquitin, kunitz-type domain of human protease inhibitors, a PDZ domain, a scorpion toxin, or a peptide aptamer. In some aspects of these embodiments, the antigen-binding site has specificity for more than one antigen. In additional aspects of these embodiments of the invention, diversification of the introduced region by V(D)J recombination and/or gene conversion in B lymphocytes results in diversification of the antigen binding sites associated with the protein scaffolds encoded by the region. In some aspects, the protein scaffolds encoded by the introduced region are expressed within chimeric immunoglobulin molecules, and in some aspects, the expressed chimeric immunoglobulin molecules comprise antigen-binding scaffold domains replacing the immunoglobulin variable domains. In some aspects, the expressed scaffold domain-containing chimeric immunoglobulin molecule is comprised of immunoglobulin heavy chain constant domains, and in some aspects, the expressed scaffold domain-containing chimeric immunoglobulin molecule is comprised of immunoglobulin heavy chain constant domains that do not require association with immunoglobulin light chains for expression on the lymphocyte surface or secretion from lymphocytes. In some aspects, one antigen-binding site has specificity for a first epitope on an antigen and another antigen-binding site has specificity for a second epitope on the same antigen; in other aspects, one antigen-binding site has specificity for a first epitope on an antigen and another antigen-binding site has specificity for a second epitope on a different antigen.

It is an object of the invention to provide non-human vertebrate cells and non-human transgenic mammals comprising an introduced nucleic acid sequence that is capable of participating in the V(D)J rearrangement and/or gene conversion process that normally diversifies antibody genes. Due to the presence of the introduced nucleic acid sequence, the modified genomic locus is capable of expressing chimeric immunoglobulin molecules comprised of scaffold domains that have antigen-binding properties. These scaffold domains comprise biologic therapeutics that may be used as an alternative to or in addition to traditional monoclonal antibodies known in the art.

Further, it is an object to provide B-cells from transgenic animals that are capable of expressing the biologic therapeutics of the invention comprising scaffold domains, where such B-cells are immortalized to provide a source of the biologic therapeutics that specifically bind to a particular antigen.

It is yet another object to provide biologic therapeutics comprising one or more scaffold domains cloned from B cells for use in the production and/or optimization of the biologic therapeutics of the invention for research tool, diagnostic and therapeutic uses.

It is a further object of the invention to provide hybridoma cells that are capable of producing biologic therapeutics comprising one or more scaffold domains.

These and other aspects, objects and features are described in more detail below.

### BRIEF DESCRIPTION OF THE FIGURES

FIG. 1 is a flow chart showing the steps for creation of a cell comprising a chimeric immunoglobulin of the invention using a strategy that includes use of recombinase-mediated deletion of a variable region of an endogenous immunoglobulin gene, and recombinase-mediated cassette exchange as a means for inserting scaffold-encoding gene segments into the modified immunoglobulin gene.

FIG. 2 is a schematic diagram illustrating the introduction of a chimeric immunoglobulin region into the genome of a non-human mammalian cell via recombinase-mediated cassette exchange.

FIG. 3 is an illustration of the structure of an adnectin domain showing the protein loops that can participate in antigen binding.

FIG. 4 is a schematic diagram illustrating one possible (2-step) DNA rearrangement event of gene segments encoding parts of adnectin domains. The arrangement of gene segments allows for extensive diversification (by DNA rearrangement) of the adnectin “FG” loop.

FIG. 5 illustrates the structure of an mRNA generated from the rearranged gene of FIG. 4.

FIG. 6 is yet another schematic diagram illustrating a possible (2-step) DNA rearrangement event of gene segments encoding parts of adnectin domains. The arrangement of gene segments allows for extensive diversification (by DNA rearrangement) of the adnectin “BC” and “FG” loops.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “antigen-binding site” refers to a site in a protein domain that is capable of specifically binding to an antigen or antigens. Preferably, the antigen-binding site binds to antigen with a Kd of at least 1 mM, for example a Kd of 10 nM, 1 nM, 500 pM, 200 pM, 100 pM, to each antigen.

The term “chimeric” as used herein in respect to nucleic acids such as DNA means two or more genetic components that are not normally found next to each other.

A “domain” is a folded protein structure that has tertiary structure independent of the rest of the protein. Generally, domains are responsible for discrete functional properties of proteins and in many cases may be added, removed or transferred to other proteins without loss of function of the remainder of the protein and/or of the domain.

The term “homology targeting vector” (or simply, “targeting vector”) refers to a vector comprising a nucleic acid encoding a targeting sequence, a site-specific recombination site, and optionally a selectable marker gene, which is used to modify an endogenous immunoglobulin region using homology-mediated recombination in a host cell. For example, a homology targeting vector can be used in the present invention to introduce a site-specific recombination site into particular region of a host cell genome.

The term “immunoglobulin variable region” as used herein refers to a nucleotide sequence that encodes all or a portion of a variable region of an antibody molecule or all or a portion of a regulatory nucleotide sequence that controls expression of an antibody molecule. Immunoglobulin regions for heavy chains may include but are not limited to all or a portion of the V, D, J, and switch regions, including introns. Immunoglobulin variable region for light chains may include but are not limited to the V and J regions, their upstream flanking sequences, introns, associated with or adjacent to the light chain constant region gene.

The term “scaffold protein”, as used herein, are carrier proteins for the display of antigen-binding sites. Protein scaffolds provide elements of secondary structure, and protein frameworks supporting one or more antigen-binding sites in a fixed spatial arrangement.

The term “research tool” as used herein refers to any composition or assay of the invention used for scientific enquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

“Site-specific recombination” refers to a process of recombination between two compatible recombination sites including any of the following three events: a) deletion of a preselected nucleic acid flanked by the recombination sites; b) inversion of the nucleotide sequence of a preselected nucleic acid flanked by recombination sites, and c) reciprocal exchange of nucleic acid regions proximate to recombination sites located on different nucleic acid molecules. It is to be understood that this reciprocal exchange of nucleic acid segments results in an integration event if one or both of the nucleic acid molecules are circular.

The term “targeting sequence” refers to a sequence that is homologous to DNA sequences in the genome of a cell that flank or occur adjacent to the region of an immunoglobulin genetic locus that is to be modified. The flanking or adjacent sequence may be within the locus itself or upstream or downstream of coding sequences in the genome of the host cell. Targeting sequences are inserted into recombinant DNA vectors for use in cell transfections such that sequences to be inserted into the cell genome, such as the sequence of a recombination site, are flanked by the targeting sequences of the vector.

The term “transgene” is used herein to describe genetic material that has been or is about to be artificially inserted into the genome of a cell, and particularly a cell of a vertebrate host animal. The term “transgene” as used herein refers to a chimeric nucleic acid, e.g., a nucleic acid in the form of an expression construct and/or a targeting vector.

By “transgenic animal” is meant a non-human animal, usually a mammal, having an exogenous nucleic acid sequence present as an extrachromosomal element in a portion of its cells or, more typically, stably integrated into its germ line DNA (i.e., in the genomic sequence of most or all of its cells). In the present invention, a chimeric nucleic acid is introduced into the germ line of such transgenic animals by genetic manipulation of, for example, embryos or embryonic stem cells of the host animal according to methods known in the art.

A “vector” includes plasmids and viruses and any DNA or RNA molecule, whether self-replicating or not, that can be used to transform or transfect a cell.

### DETAILED DESCRIPTION OF THE INVENTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include construction and breeding of transgenic animals, hybridization and ligation of polynucleotides, and recombination of polynucleotides. Specific illustrations of suitable techniques can be had by reference to the examples herein. However, other equivalent conventional procedures can, of course, also be used. Such conventional techniques and descriptions can be found in standard laboratory manuals such as Butler (2004), Animal Cell Culture (BIOS Scientific); Picot (2005), Human Cell Culture Protocols (Humana Press), Davis (2002), Basic Cell Culture, Second Ed. (Oxford Press); Lanza, et al., (Eds.) (2009), Essentials of Stem Cell Biology, Second Ed. (Elsevier Academic Press); Lanza, (Ed.) (2009), Essential Stem Cell Methods (Elsevier Academic Press); and Loring, et al. (Eds.) (2007), Human Stem Cell Manual (Elsevier Academic Press); Freshney (2010), Culture of Animal Cells (John Wiley & Sons); Ozturk and Hu (2006), Cell Culture Technology for Phamaceutical and Cell-Based Therapies (CRC Press); Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W.H. Freeman Pub., New York, N.Y.; and Berget al. (2002) Biochemistry, 5th Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a locus” refers to one or more loci, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### The Invention in General

The present invention provides non-human host cells and animals for the identification and production of biologic therapeutics that selectively bind to one or more antigens of interest. Although such engineered molecules have enjoyed impressive recent successes in yielding clinical candidates—including some that are currently being tested in clinical trials—the in vitro procedures to identify and develop these molecules fall short of replicating the power of an in vivo immune system in terms of coupling a highly effective capability for diversification with rapid and similarly effective selection for optimal target-binding properties. The present invention addresses this deficiency by engineering a vertebrate immune system such that it expresses the biologic therapeutics instead of antibodies.

Importantly, the engineering permits large-scale diversification of the biologics by a genomic rearrangement process similar to the natural one that diversifies antibodies. It also permits secondary diversification by the very powerful natural process of somatic hypermutation (i.e., affinity maturation).

The present invention provides non-human vertebrate cells comprising an introduced chimeric region comprising exogenous nucleic acids encoding protein scaffolds. The region also comprises noncoding sequences that include regulatory regions that influence the expression of the scaffold-encoding nucleic acid. The noncoding sequences may be derived from the host animal, or other animals, or may be partially or entirely designed—that is artificial—in nature. This chimeric region allows the transgenic animal to produce diverse antigen-binding scaffold-containing proteins, while the noncoding sequences help to promote efficient expression of the scaffold-encoding genes in the desired B lymphocyte cell types. The present invention comprises the use of a synthetic or recombinantly produced chimeric region comprising both protein scaffold structures and non-coding sequences.

The present invention provides a number of advantages over existing strategies for diversifying biologic therapeutics such as adnectins or anticalins. Conventional methods involve creating libraries of genes in which the sequences encoding the antigen-binding surface-exposed peptide loops have been mutagenized in a random (or semi-random) fashion. Utilizing in vivo V(D)J rearrangement provides an alternative means for generating diversity featuring loop replacement coupled with residue addition or deletion at the junctions between the loops and the domain framework. The use of D-like and J-like gene segments also allows for amino acids to be added or deleted semi-randomly within the loops and/or at their ends.

In preferred aspects of the invention, this chimeric region is introduced into a rodent host vertebrate cell. In some instances, however, it may be desirable to use another host, such as an avian host. In a specific aspect of the invention, the transgenic non-human mammal comprises an introduced nucleic acid comprising multiple scaffold-encoding gene segments with noncoding sequences derived from immunoglobulin genes in non-human mammal host.

The methods of the invention utilize a combination of homologous recombination and site-specific recombination to create the cells and animals of the invention. In a preferred version of the invention, homology targeting vectors are first used to introduce site-specific recombination sites into the host mammal genome at the desired location in an endogenous immunoglobulin locus.

Exemplary methodologies for homologous recombination are described in U.S. Pat. Nos. 6,689,610; 6,204,061; 5,631,153; 5,627,059; 5,487,992; and 5,464,764, each of which is incorporated by reference in its entirety. Thus, in specific aspects of the invention, a homology targeting vector can be utilized to replace certain sequences within the endogenous genome as well as introducing the site-specific recombination sites and selectable markers.

The modified locus can be created using various conventional techniques, as will be obvious to one skilled in the art upon reading the present disclosure. Preferably, the modified locus is generated by inserting a piece of DNA (referred to here as the “donor DNA”) containing gene segments encoding parts of one or more scaffold domains adjoined to an array of D-like and J-like gene segments directly into a modified version of a mouse immunoglobulin locus such as the mouse heavy chain locus (referred to here as the “acceptor allele”). The acceptor allele lacks all of the endogenous variable, diversity and joining gene segments. It instead contains recognition sites for the Cre recombinase (a loxP site and a mutated version of the loxP site). The donor DNA is flanked by the same Cre recombinase recognition sites (i.e., on one side there is a loxP site and on the other there will be a mutated version of the loxP site). The Cre recombinase is used to catalyze the insertion of the donor DNA into the acceptor allele. In a preferred version of the invention, the donor DNA includes constant domain-encoding exons.

The synthetic chimeric immunoglobulin region comprising the scaffold-encoding gene segments preferably includes customized selection markers to facilitate the recombinase-mediated cassette exchange process used for high efficiency insertion of the region into the desired antibody gene. The selection markers (flanked by recognition sites for a site-specific recombinase) are optionally excised in a following step, e.g., during the breeding of ES cell-derived mice to mice expressing the relevant site-specific recombinase in their germlines.

In an alternative version of the invention, the scaffold-encoding gene segments are introduced into an immunoglobulin locus primarily, if not exclusively, by homologous recombination. In such a version, targeting vectors are employed that are comprised of genomic targeting homology arms flanking a nucleic acid sequence comprising scaffold-encoding gene segments. These genomic homology arms facilitate insertion of the scaffold-encoding DNA into an immunoglobulin locus, such as that which encodes the immunoglobulin heavy chain. In a preferred such embodiment of the invention, one or more targeting vectors are employed to insert the scaffold-encoding DNA into the variable region of the heavy chain locus. In a further preferred embodiment of the invention, the insertions are conducted at both ends of the variable region (i.e., at the distal end of the variable gene segment array, and at the proximal end of the J gene segment array, where proximity is defined relative to the constant domain exons in the locus). Multiple independent insertions may be required to deliver all of the desired scaffold-encoding gene segments. As in other embodiments of the invention, deletion of the endogenous variable region of the immunoglobulin locus may be performed, and if so, this may be accomplished directly by homologous recombination. Since the latter form of deletion by homologous recombination is expected to be inefficient given the large size of the variable region, site-specific recombination optionally may be employed to accomplish this step. Such site-specific recombination depends on insertion of recognition sites for the recombinase of choice (e.g., Cre or Flp) during the preceding homologous recombination steps in which the scaffold-encoding DNA was inserted at both ends of the variable region. Positive or negative selection may be employed to facilitate isolation of the desired genome comprising the deleted and modified (i.e., scaffold-encoding) immunoglobulin locus.

In many instances in which homologous recombination is employed to accomplish a genetic change in a genome—be it an insertion or a deletion—a further modification of the invention would involve the use of engineered site-specific endonucleases to increase the likelihood that a desired outcome can be accomplished. Such endonucleases are of value because they can be engineered to be highly specific for unique sequences in a target genome, and because they cause double-stranded DNA breaks at the sites they recognize. Double-stranded breaks promote homologous recombination with targeting vectors that carry targeting homology with DNA in the immediate vicinity of the breaks. Thus, the combination of a targeting vector and a site-specific endonuclease that cleaves DNA within or close to the region targeted by a vector typically results in much higher homologous recombination efficiency than use of a targeting vector alone. Furthermore, it is possible to facilitate the creation of a genomic deletion through use of one or more site-specific endonucleases and a targeting vector comprised of two targeting homology arms in which one arm targets one side of the region to be deleted and the other arm targets the other side.

### Protein Scaffolds

In specific aspects, the protein scaffold used in the chimeric immunoglobulin of the invention is derived from a scaffold or a derivative of a scaffold selected from the group consisting of CTLA-4 (Evibody); lipocalin; Protein A derived molecules such as Z-domain of Protein A (Affibody, SpA); Adomain (Avimer/Maxibody); Heat shock proteins such as GroEl and GroES; transferrin (trans-body); ankyrin repeat protein (DARPin); peptide aptamer; Ctype lectin domain (Tetranectin); human gamma-crystallin and human ubiquitin (affilins); PDZ domains; scorpion toxin kunitz type domains of human protease inhibitors; and fibronectin (adnectin).

CTLA-4 (Cytotoxic T Lymphocyte-associated Antigen 4) is a CD28-family receptor expressed on activated T cells. Its extracellular domain has a variable domain-like Ig fold. Loops corresponding to complementarity determining regions of antibodies can be substituted with heterologous sequence to confer different binding properties. CTLA-4 molecules engineered to have different binding specificities are also known as Evibodies. For further details see Journal of Immunological Methods, 248(1-2):31-45 (2001).

Lipocalins are a family of extracellular proteins which transport small hydrophobic molecules such as steroids, bilins, retinoids and lipids. They have a rigid 13-sheet secondary structure with a number of loops at the open end of a conical structure that is engineered to bind to different target antigens. Anticalins are between 160-180 amino acids in size, and are derived from lipocalins. For further details see Biochim Biophys Acta, 1482: 337-350 (2000), U.S. Pat. No. 7,250,297B1 and US Pub. No. 20070224633.

An affibody is a scaffold derived from Protein A of Staphylococcus aureus that is engineered to bind to antigen. The domain consists of a three-helical bundle of approximately 58 amino acids. Libraries have been generated by randomization of surface residues. For further details see Protein Eng. Des. Sel., 17:455-62 (2004) and EP1641818A1.

Avimers are multidomain proteins derived from the A-domain scaffold family. The native domains of approximately 35 amino acids adopt a defined disulphide-bonded structure. Diversity is generated by shuffling of the natural variation exhibited by the family of A-domains. For details see Nature Biotechnology, 23(12):1556-61 (2005) and Expert Opinion on Investigational Drugs, 16(6):909-17 (2007).

A transferrin is a monomeric serum transport glycoprotein. Transferrins are engineered to bind different target antigens by insertion of peptide sequences in a permissive surface loop. Examples of engineered transferrin scaffolds include the Trans-body. For further details see J. Biol. Chem., 274:24066-73 (1999).

Designed Ankyrin Repeat Proteins (DARPins) are derived from Ankyrins, which are a family of proteins that mediate attachment of integral membrane proteins to the cytoskeleton. A single ankyrin repeat is a 33 residue motif consisting of two a-helices and a β-turn. Ankyrins can be engineered to bind different target antigens by randomizing residues in the first alpha-helix and a β-turn of each repeat. Their binding interface can be varied by altering the number and nature of the modules included in the scaffold protein (a method of affinity maturation). For further details see J. Mol. Biol., 332:489-503 (2003), PNAS, 100(4):1700-05 (2003) and J. Mol. Biol., 369:1015-28 (2007) and US Pub. No. 20040132028A1.

Adnectins consist of a backbone of the natural amino acid sequence of the 10th domain of the 15 repeating units of human fibronectin type III (FN3). Three loops at one end of the β-sandwich are engineered to enable an Adnectin to specifically recognize a therapeutic target of interest. For further details see Protein Eng. Des. Sel., 18:435-44 (2005), U.S. Pat. No. 20080139791, WO2005056764 and U.S. Pat. No. 6,818,418B1.

Peptide aptamers are combinatorial recognition molecules that consist of a constant scaffold protein, typically thioredoxin (TrxA) that contains a constrained variable peptide loop inserted at the active site. For further details see Expert Opin. Biol. Ther., 5:783-97 (2005).

Microbodies are derived from naturally occurring microproteins of 25-50 amino acids in length that contain 3-4 cysteine bridges; examples of microproteins include KalataB 1 and conotoxin and knottins. The micro proteins have a loop that is engineered to include up to 25 amino acids without affecting the overall fold of the micro protein. For further details of engineered knottin domains, see WO2008098796.

Other antigen-binding scaffold proteins include human γ-crystallin and human ubiquitin (affilins), kunitz type domains of human protease inhibitors, PDZ-domains of the Ras-binding protein AF-6, scorpion toxins (charybdotoxin), and C-type lectin domain (tetranectins) are reviewed in Chapter 7, Non-Antibody Scaffolds from Handbook of Therapeutic Antibodies (2007, edited by Stefan Dubel) and Protein Science, 15:14-27 (2006). Antigen-binding scaffolds of the present invention could be derived from any of these alternative protein domains.

### Site-Specific Recombination

Site-specific recombination differs from general homologous recombination in that short specific DNA sequences, which are required for the recombinase recognition, are the only sites at which recombination occurs. Site-specific recombination requires specialized recombinases to recognize the sites and catalyze the recombination at these sites. A number of bacteriophage and yeast-derived site-specific recombination systems, each comprising a recombinase and specific cognate sites, have been shown to work in eukaryotic cells for the purpose of DNA integration and are therefore applicable for use in the present invention. These include the bacteriophage P1 Cre/lox, yeast FLP-FRT system, and the Dre system of the tyrosine family of site-specific recombinases. Such systems and methods of use are described, for example, in U.S. Pat. Nos. 7,422,889; 7,112,715; 6,956,146; 6,774,279; 5,677,177; 5,885,836; 5,654,182; and 4,959,317, which are incorporated herein by reference to teach methods of using such recombinases. The recombinase-mediated cassette exchange (RMCE) procedure is facilitated by usage of the combination of wild-type and mutant loxP (or FRT etc) sites together with the appropriate recombinase (e.g., Cre or Flp), and negative and/or positive selection. RMCE will occur when the sites employed are identical to one another (and not, as just stated a combination of wild-type and mutant sites) and/or in the absence of selection, but the efficiency of the process is reduced because excision rather than insertion reactions are favored, and (without incorporating positive selection) there will be no enrichment for appropriately mutated cells.

Other systems of the tyrosine family such as bacteriophage lambda Int integrase, HK2022 integrase, and in addition systems belonging to the separate serine family of recombinases such as bacteriophage phiC31, R4Tp901 integrases are known to work in mammalian cells using their respective recombination sites, and are also applicable for use in the present invention.

The methods of the invention preferably utilize site-specific recombination sites that utilize the same recombinase, but which do not facilitate recombination between the sites. For example, a loxP site and a mutated loxP site can be integrated into the genome of a host, but introduction of Cre into the host will not cause the two sites to undergo recombination; rather, the loxP site will recombine with another loxP site, and the mutated site will only recombine with another likewise mutated loxP site.

Two classes of variant recombinase sites are available to facilitate recombinase-mediated cassette exchange. One harbors mutations within the 8 bp spacer region of the site, while the other has mutations in the 13-bp inverted repeats.

Spacer mutants such as lox511 (Hoess, et al., Nucleic Acids Res., 14:2287-00 (1986)), lox5171 and lox2272 (Lee and Saito, Gene, 216:55-65 (1998)), m2, m3, m7, and mil (Langer, et al., Nucleic Acids Res., 30:3067-77 (2002)) recombine readily with themselves but have a markedly reduced rate of recombination with the wild-type site. Examples of the use of mutant sites of this sort for DNA insertion by recombinase-mediated cassette exchange can be found in Baer and Bode, Curr Opin Biotechnol, 12:473-80 (2001); Albert et al., Plant J., 7:649-59 (1995); Seibler and Bode, Biochemistry, 36:1740-47 (1997); Schlake and Bode, Biochemistry, 33:12746-51 (1994).

Inverted repeat mutants represent a second class of variant recombinase sites. For example, loxP sites can contain altered bases in the left inverted repeat (LE mutant) or the right inverted repeat (RE mutant). An LE mutant, lox71, has 5 bp on the 5′ end of the left inverted repeat that is changed from the wild type sequence to TACCG (Araki, Nucleic Acids Res., 25:868-72 (1997)). Similarly, the RE mutant, lox66, has the five 3′-most bases changed to CGGTA. Inverted repeat mutants can be used for integrating plasmid inserts into chromosomal DNA. For example, the LE mutant can be used as the “target” chromosomal loxP site into which the “donor” RE mutant recombines. After recombination a donor piece of DNA that contained an RE site will be found inserted in the genome flanked on side by a double mutant site (containing both the LE and RE inverted repeat mutations) and on the other by a wild-type site (Lee and Sadowski, Prog Nucleic Acid Res Mol Biol., 80: 1-42 (2005); Lee and Sadowski, J Mol Biol., 326:397-412 (2003)). The double mutant is sufficiently different from the wild-type site that it is unrecognized by Cre recombinase and the inserted segment therefore cannot be excised by Cre-mediated recombination between the two sites.

In certain aspects, site-specific recombination sites can be introduced into introns or intergenic regions, as opposed to coding nucleic acid regions or regulatory sequences. This may avoid inadvertently disrupting any regulatory sequences or coding regions necessary for proper gene expression upon insertion of site-specific recombination sites into the genome of the animal cell.

Introduction of the site-specific recombination sites may be achieved by conventional homologous recombination techniques. Such techniques are described in references such as e.g., Sambrook and Russell (2001) Molecular cloning: a laboratory manual, 3d ed. (Cold Spring Harbor, N.Y.: Cold Spring Harbor Laboratory Press); Nagy, (2003) Manipulating the mouse embryo: a laboratory manual, 3d ed. (Cold Spring Harbor, N.Y.: Cold Spring Harbor Laboratory Press); and Miller, Vandome, and McBrewster (2009) Genetic Recombination: Nucleic acid, Homology (biology), Homologous recombination, Non-homologous end joining, DNA repair, Bacteria, Eukaryote, Meiosis, Adaptive immune system, V(D)J recombination.

Specific recombination into the genome can be facilitated using vectors designed for positive or negative selection as known in the art. In order to facilitate identification of cells that have undergone the replacement reaction, an appropriate genetic marker system may be employed and cells selected by, for example, use of a selection medium. However, in order to ensure that the genome sequence is substantially free of extraneous nucleic acid sequences at or adjacent to the two end points of the replacement interval, desirably the marker system/gene can be removed following selection of the cells containing the replaced nucleic acid.

In one preferred aspect of the methods of the present invention, cells in which the replacement of all or part of the endogenous immunoglobulin has taken place are negatively selected upon exposure to a toxin or drug. For example, cells that retain expression of HSV-TK can be selected through use of appropriate use of nucleoside analogues such as gancyclovir. In another aspect of the invention, cells comprising the deletion of the endogenous immunoglobulin region may be positively selected by use of a marker gene, which can optionally be removed from the cells following or as a result of the recombination event. A positive selection system that may be used is based on the use of two non-functional portions of a marker gene, such as HPRT, that are brought together through the recombination event. These two portions are brought into functional association upon a successful replacement reaction being carried out and wherein the functionally reconstituted marker gene is flanked on either side by further site-specific recombination sites (which are different to the site-specific recombination sites used for the replacement reaction), such that the marker gene can be excised from the genome, using an appropriate site-specific recombinase.

The recombinase may be provided as a purified protein, or may be expressed from a construct transiently expressed within the cell in order to provide the recombinase activity. Alternatively, the cell may be used to generate a transgenic animal, which may be crossed with an animal that expresses said recombinase, in order to produce progeny that lack the marker gene and associated recombination sites.

### Generation of Transgenic Animals

In specific aspects, the invention provides methods for the creation of transgenic animals comprising the introduced chimeric immunoglobulin region.

In one aspect, the host cell utilized for replacement of the endogenous immunoglobulin genes is an embryonic stem (ES) cell, which can then be utilized to create a transgenic mammal. Thus, in accordance with one aspect, the methods of the invention further comprise: isolating an embryonic stem cell that comprises the introduced chimeric immunoglobulin region and using said ES cell to generate a transgenic animal that contains the replaced immunoglobulin locus.

In another example, the transgenic animal is avian, and the animal is produced using primordial germ cells. Thus, in accordance with another aspect, the methods of the invention further comprise: isolating a primordial germ cell that comprises the introduced chimeric immunoglobulin region and using said germ cell to generate a transgenic animal that contains the replaced immunoglobulin locus. Methods for production of such transgenic avians are disclosed, e.g., in U.S. Pat. Nos. 7,323,618 and 7,145,057, which are incorporated herein by reference.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to terms and numbers used (e.g., vectors, amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Introduction of a Chimeric Immunoglobulin Region into the V_{H} Gene Locus of a Mouse Genome

A method for replacing a portion of a mammalian genome with chimeric immunoglobulin region comprising adnectin or anticalin scaffold-encoding gene segments is illustrated in FIGS. 1-7. FIG. 1 shows a flow chart illustrating the different steps of this aspect of the invention. The method provides introducing two site-specific recombination sites into the host genome. Preferably this is accomplished by introducing a first site-specific recombination site into the mammalian genome, which may be introduced 5′ of the endogenous constant domain regions of the mammalian genome, followed by the introduction of a second site-specific recombination site, which in combination with the first site-specific recombination site flanks the endogenous immunoglobulin variable region. The flanked endogenous region upstream of the constant domains is deleted 102 and a synthetic nucleic acid sequence encoding one or more protein scaffold sequences and optionally one or more drug resistance is introduced 104 via recombinase-mediated cassette exchange. The cells with the introduced synthetic region are selected 106, e.g., by exposure to antibiotics that are rendered ineffectual by expression of the inserted drug resistance gene. The nucleic acid used for selection is removed 108, leaving the chimeric immunoglobulin region with the synthetic protein scaffold sequences and the endogenous constant domains.

An exemplary method illustrating the introduction of a chimeric region into the genomic locus of a mouse ES cell is illustrated in more detail in FIGS. 2-6. In FIG. 2, a vector 201 is provided comprising adnectin or anticalin gene segments 203 flanked by recombinase recognition sites 205, 207, e.g., FRT or loxP 207, for Flp and Cre, respectively. The vector comprises one or preferably two selection genes 209, 211 for use in selection of cells expressing the introduced construct in future steps. The vector may also optionally comprise a visual marker such as a fluorescent green protein (GFP) (not shown). The vector 201 is introduced 202 to a modified mouse ES cell, which has the endogenous immunoglobulin variable region deleted and replaced with recombination sites 217, which are the same as the sites 205, 207 on the vector. The site-specific recombination sites and the protein scaffold regions 203 of the targeting vector 201 are integrated 204 into the mouse genome 5′ of the endogenous mouse constant domain exons 215 to produce the chimeric immunoglobulin locus 219.

A variation on the scheme depicted in FIG. 2 involves inclusion of constant domain exons within the introduced DNA such that these exons are used preferentially in place of the endogenous ones. A further variation involves introduction of the vector 201 into the Kappa locus of the ES cell rather than the heavy chain locus; in this variation, the vector may also comprise constant domain exons from the heavy chain locus. Yet another variation involves introduction of the vector 201 into the lambda locus rather than the heavy chain locus; in this variation again the vector may comprise constant domain exons derived from the heavy chain locus.

FIG. 3 illustrates an adnectin domain, one of the various protein scaffolds that can be used to create the chimeric immunoglobulin regions for use in the invention. The diagram shows the three-dimensional structure of the adnectin domain with the location of loops that can comprise antigen-binding surfaces. Preferably, the chimeric nucleic acid sequences present in the scaffold-encoding locus are designed to allow diversification of the antigen-binding surfaces by DNA rearrangement and/or gene conversion.

FIG. 4 illustrates a strategy for such diversification of the FG loop of an adnectin domain by DNA rearrangement. The design of the chimeric immunoglobulin locus comprises multiple V-like gene segments encoding partial adnectin domains, each inclusive of the sequences specifying the BC loops 401 and DE loops 403. The locus also includes a region 405 comprised of multiple D-like 407 and J-like 409 gene segments that may be recombined to generate the FG loop with additional diversity. The chimeric immunoglobulin undergoes DJ-like rearrangement 402 (to give the partially rearranged product 411) and VJ-like rearrangement 404 (to give the fully rearranged product 413) similar to that of wild-type endogenous immunoglobulin genes. Diversity in the rearranged locus is partially contributed by N- and P-nucleotide addition/deletion during recombination 402, 404. The multiple V-like gene segments differ from one another in their BC 401 and DE 403 loop-encoding sequences and also in surrounding framework-encoding sequences. The mRNA generated from the rearranged gene including sequences encoding immunoglobulin constant domains from downstream exons is illustrated in FIG. 5. The adnectin domain 501 is provided in a genomic region upstream of the constant domains 503.

FIG. 6 illustrates an alternative strategy for the diversification of the FG loop of an adnectin domain. The design of this chimeric immunoglobulin locus comprises one or more V-like gene segments encoding a small fraction of the domain coding information for beta strands A and B. The chimeric locus undergoes DJ-like rearrangement 602 and VJ-like rearrangement 604 similar to that of wild-type endogenous immunoglobulins. In contrast to the design shown in FIG. 4, the majority of the domain coding information is present within expanded D-like gene segments, which are each inclusive of loops BC 601 and DE 603. This arrangement of gene segments allows for diversification of an end of the BC loop during joining 604 between the D-like and V-like gene segments. The FG loop is also diversified during DJ-like rearrangement 602 between the expanded D-like gene segments and the J-like gene segments 605.

The primary screening for introduction of the chimeric immunoglobulin region is carried out by Southern blot, or with primary polymerase chain reaction (PCR) screens supported by secondary screens with Southern and/or loss-of-native-allele quantitative PCR screens.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. §112, ¶6.

1-22. (canceled)

23. A transgenic non-human vertebrate with a genome comprising an engineered locus comprised of introduced gene segments structured and organized in such a fashion that they are permissive of RAG-1/RAG-2-mediated gene rearrangement, with said rearrangement having the capacity to create diversity in a polypeptide-coding region derived in part or entirely from the rearranged gene segments, with said created diversity allowing the encoded polypeptides to bind diverse antigens, and with said diversified polypeptide sequence not encoding a native antibody variable domain.

24. The transgenic non-human vertebrate of claim 23, wherein the diversified polypeptide sequence is derived from CTLA-4, lipocalin, anticalin, Protein A, an affibody, Protein G, fibronectin, adnectin an A-domain, a Heat shock protein, GroEI, GroES, transferring, ankyrin, a C-type lectin domain, human γ-crystallin, human ubiquitin, kunitz-type domain of human protease inhibitors, a PDZ domain, a scorpion toxin, or a peptide aptamer.

25. The transgenic non-human vertebrate of claim 23, wherein the introduced gene segments are structured and arranged in such a fashion that they can be diversified by gene conversion.

26. The transgenic non-human vertebrate of claim 23, wherein the engineered immunoglobulin locus is an endogenous immunoglobulin-encoding locus deleted of some, or all, of its variable region-encoding gene segments.

27. The transgenic non-human vertebrate of claim 23, wherein the diversified polypeptide coding region is operatively linked with the open reading frame for one or more immunoglobulin constant domains, such that the protein products of the engineered locus have a chimeric nature.

28. The transgenic non-human vertebrate of claim 27, wherein the chimeric protein product is comprised of immunoglobulin heavy chain constant domains.

29. The transgenic non-human vertebrate of claim 28, wherein the heavy chain constant domains do not require association with immunoglobulin light chains for efficient expression on the surface of lymphocytes or for efficient secretion from lymphocytes.

30. The transgenic non-human vertebrate of claim 27, wherein the chimeric protein product is comprised of immunoglobulin light chain constant domains.
